(19) United States
(12) Patent Application Publication
(10) Pub. No.: US 2024/0335148 A1
(43) Pub. Date: Oct. 10, 2024

(54) WIRELESS STICKER TYPE ANTENNA FOR REMOTE SENSING OF URINE ACTIVITY

(71) Applicant: Purdue Research Foundation, West Lafayette, IN (US)

(72) Inventors: Rahim Rahimi, West Lafayette, IN (US); Sarath Gopalakrishnan, West Lafayette, IN (US); Muhammad Masud Rana, West Lafayette, IN (US)

(73) Assignee: Purdue Research Foundation, West Lafayette, IN (US)

(21) Appl. No.: 18/631,886

(22) Filed: Apr. 10, 2024

Related U.S. Application Data
(60) Provisional application No. 63/458,224, filed on Apr. 10, 2023.

(51) Int. Cl. A61B 5/20; G01F 23/26; G01N 27/07; G01N 33/493; H01Q 1/38

(52) U.S. Cl. CPC A61B 5/20; G01F 23/26; G01N 27/07; G01N 33/493; H01Q 1/38

(57) ABSTRACT

A system for non-invasive urinary activity monitoring may include a sticker that attaches to a urine bag. The sticker may include a metal layer shaped as an antenna. A device may cause a network analyzer to send a signal in a direction of the urine bag. The device may detect a shift in a resonant frequency of the antenna. The device may measure, based on the change in resonant frequency, an amount of fluid in the container. The device may measure, based on a change in the resonant peak amplitude, the conductivity of the fluid in the container.

### CROSS REFERENCE TO RELATED APPLICATIONS

This application claims the benefit of U.S. Provisional Application No. 63/458,224 filed Apr. 10, 2023, the entirety of which is hereby incorporated by reference.

### TECHNICAL FIELD

This disclosure relates to medical devices and, in particular, to monitoring fluidic activity and properties in reservoirs.

### BACKGROUND

In the last decade, the rate of hospitalization of critically ill patients has sharply increased which reached an all-time high during COVID-19. About 5 million critically ill patients are admitted annually to U.S. intensive care units (ICUs) to prevent life-threatening clinical conditions, such as physiological deterioration, respiratory failure, acute myocardial infarction, cerebral infarction/intracranial hemorrhage, and sepsis, which requires constant care, close supervision, and around-the-clock monitoring. Among the critically ill patients in ICUs, 45-79% use a urinary catheter due to immobilization, urinary retention, urinary incontinence, or post-surgery care. While urinary catheters significantly assist proper bladder voiding in patients, widespread usage of catheters in ICUs poses two main challenges: one, the requirement of frequent monitoring and draining of the urine bag, and two, the increased risk of infection through catheters. Proper management of urinary catheters and urine bags often requires visual inspection by healthcare technicians which increases workload, fatigue, and human errors, and as observed during COVID-19, rapidly increases the exposure of healthcare technicians to contagious patients. Besides the complications with respect to frequency monitoring, the urinary catheterization procedure is prone to a high risk of infection as it introduces a foreign object into the body which permits opportunistic bacteria to enter the urinary tract of the patient. Consequently, about 70% of catheterized patients are afflicted with some degree of urinary tract infection (UTI) which can lead to complications starting from pyelonephritis to life-threatening clinical conditions, such as renal failure and sepsis. However, early detection of UTI can help prevent the aggravation of the infection from benign symptoms to clinical complications through the timely replacement of urinary catheters and prophylactic antibiotic administration.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 illustrates an example of a system for non-invasive urinary activity monitoring.

FIG. 2 illustrates a disposable antenna sticker at various stages of manufacture.

FIG. 3 illustrates a block diagram of electronic components for a portable remote monitoring device.

FIG. 4 illustrates a second example of a system with a urine container and remote monitoring device.

FIG. 5 illustrates results demonstrating a shift in the resonant frequency characteristics for different values of fluid volume.

FIG. 6 illustrates results demonstrating the shift in the amplitude of the resonant peak for different values of urine conductivity.

### DETAILED DESCRIPTION

Early detection of UTI can be achieved by monitoring urinary frequency and fluctuations in the chemical compositions and properties of urine (caused by changes in concentration of ions and microbial action) often manifested through various known biomarkers, such as pH, nitrite, conductivity, and leukocyte esterase. Despite this critical need to monitor these parameters frequently and non-invasively, most previous methods to analyze these markers are based on discrete, invasive measurement methods which can be broadly classified into colorimetric techniques and culture assay-based urinalysis. The colorimetric techniques rely on dipsticks coated with stimuli-sensitive chemicals that show a visual response to elevated levels of UTI indicators, such as pH, nitrite, or blood. However, colorimetric techniques rely heavily on visual inspection which lacks quantitative analysis and is, therefore, error-prone and inaccurate. Furthermore, colorimetric techniques envisage limitations in estimating the degree of infection as it is a discrete measurement method. In addition, this technique requires direct contact with the specimens which can lead to test fluid contamination as well as a high risk of exposure of the technician to potentially infected specimens. On the other hand, culture assay can be used to accurately quantify the number and type of microorganisms in the urine and has been widely used as the gold standard test for the diagnosis of UTI. Although cultural assays have been widely used as part of urinalysis in the healthcare sector, they are labor intensive, invasive, and require carefully procured urine culture in a lab with manual assistance. Moreover, these techniques have a turnaround time of at least 48 h and mandate proper storage and transportation in order to obtain valid and reliable test results. Such prolonged turnaround times can expedite the spread of infection to the crucial parts of the urinary tract in infected patients and can cause substantially delayed diagnosis and inflated treatment complications such as sepsis and morbidity. As an ideal proposition, early detection of UTI using a contactless real-time monitoring system with wireless data transmission capabilities can help prevent potentially life-threatening complications while averting the risk of exposure of healthcare providers to patients. However, existing systems are not equipped to address the vitally important requirement of in-situ real-time detection of UTI in catheterized patients in ICUs.

To address these and other challenges, provided herein is a multi-functional contactless remote monitoring system to measure urine level and urine conductivity as markers of risk of infection in catheterized patients. The remote monitoring system allows detection of not only the risk of infection but also the timely fill status of the urine bags, thus providing effective management of the draining of the urine bags which in turn helps in reducing the labor and burden of healthcare providers as well as their risk of exposure to infected patients. This device is composed of a low-cost disposable module and a reusable electronic module. The disposable module is implemented as a simple sticker antenna and is interfaced with the reusable electronic module comprising of a custom-designed portable remote monitoring system. The sticker antenna allows easy attachment to any number of urine bags providing disposability of the sensing units and easy applicability on different urine bag models and types. The sticker antenna may include, in some examples, two pairs of parallel plate resonators, one for urine level detection and the other for urine conductivity detection. The parallel plate resonator designed for level detection demonstrates a shift in the resonant frequency proportional to the urine volume in the bag. On the other hand, the parallel plate resonator designed for conductivity detection demonstrates a change in the amplitude of the resonant peak with respect to the attenuation caused by the conductivity variations in the medium surrounding the resonator. The required resonant frequency and attenuation are recorded by the portable remote monitoring system using an embedded network analyzer and are easily transmitted with the help of an onboard WiFi module. The integrated wireless data transmission and the remote monitoring system enable easy analysis of multiple patients at the same time. The technique reported here is a milestone in improving the quality of health provided to critically ill patients through early-stage detection of UTI in addition to drastically minimizing the risk of exposure and intensive labor of healthcare providers by tremendously enhancing medical automation.

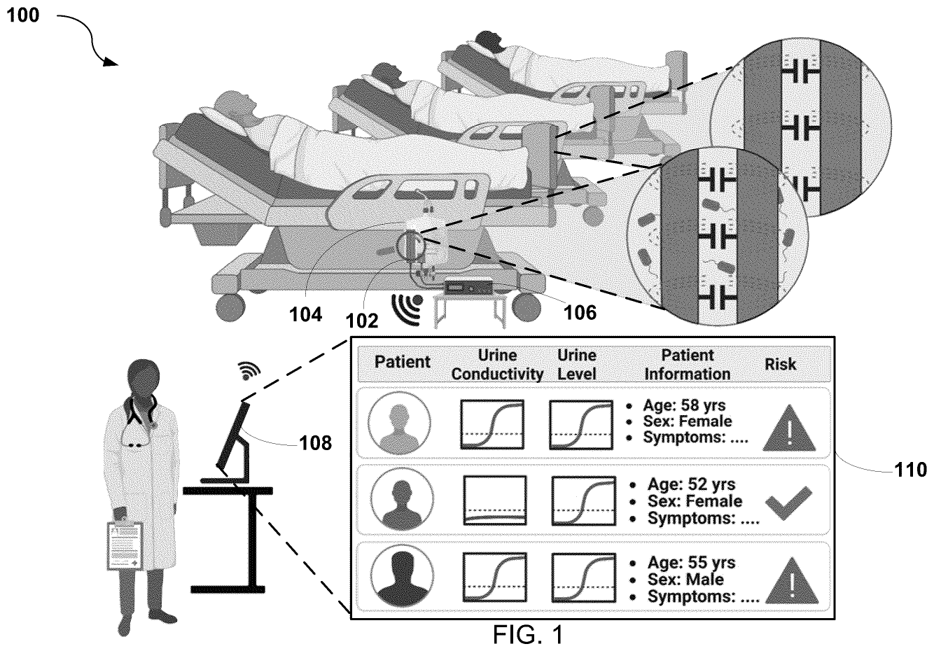

FIG. 1 illustrates an example of a system 100 for non-invasive urinary activity monitoring. The system 100 may include a disposable antenna 102 attached to a fluid bag 104 and a portable remote monitoring device 106. In various examples, the fluid bag 104 may include a urine bag. The system 100 may measure the urine volume and conductivity levels in urine bag(s) of catheterized patients. By way of example, the portable remote monitoring device 106 may perform the measurement and/or perform various operations on the measurements. Alternatively or in addition, the portable remote monitoring device may transmit the measurements to another device or a cloud device for further processing. For example, the system may include a display screen 108 and the portable remote monitoring device may transmits the recorded data to the healthcare provider's display screen for observation and diagnosis. The display screen 108, the remote monitoring device 106, or some other device which receives and/or analyzes the measurements may generate a graphical user interface 110 for displaying.

The disposable antenna 102 may be a sticker antenna that can be attached to the fluid bag. The disposable antenna comprises two parallel electrodes. By way of example, the electrodes may be plate resonators (i.e. 20 cm×2 cm and 4 cm×2 cm) capable of non-invasively measuring both the volume and conductivity of the urine inside the urine bag. The remote monitoring device 106 interfaced with the antenna may include an embedded network analyzer with wireless data transmission capabilities which enables in-situ real-time monitoring of the volume and conductivity values accessible to healthcare providers.

Various experimentation and systematic studies on the designed antennas revealed a linear and stable performance in the practically and physiologically relevant ranges of urine volume (0 to 2000 ml) and conductivity (5 mS/cm to 40 mS/cm). As a proof-of-concept, an embodiment of the system was tested in artificially created healthy and infected urine specimens to validate the system's performance in detecting the onset of UTI in catheterized patients in a hospital-like environment. STARS offers a viable solution to the emerging need for early detection of UTI and provides a steppingstone toward medical automation.

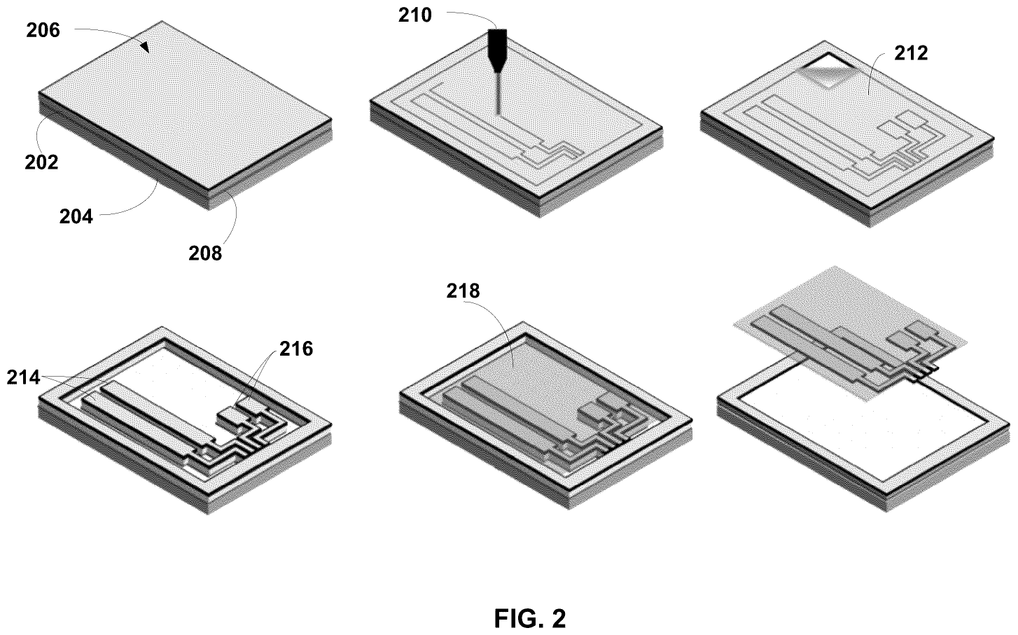

FIG. 2 illustrates a disposable antenna sticker at various stages of manufacture. The sticker antenna at the various stages of manufacture are labeled i through vi in FIG. 2. To fabricate the antenna sticker, metalized adhesive-backed labels are first provided (i). The metalized adhesive-backed labels include a face layer 202 and a release layer 204. The face layer includes a paper-based facestock with metal surface 206 (for example a laminated mental such as aluminum foil of thickness 60 μm) on the top and a bonding surface 208 made of adhesive on the bottom. The face layer is attached to the release layer. The release layer includes a release liner coated with silicone to prevent the adhesive from permanently sticking to the release liner.

The fabrication of smart stickers proceeds with laser processing of metalized adhesive-backed labels (ii). The scalable laser manufacturing technique offers rapid production of smart stickers using roll-to-roll processing. The face layer of the metalized adhesive-backed label may be patterned by a laser 210 to define the electrodes for urine level detection and urine conductivity detection in accordance with optimized dimensions of a first pair of electrodes having dimensions of 20 cm×2 cm and a second pair of electrodes having dimensions of 4 cm×2 cm, respectively. Other dimensions are possible in different embodiments.

In various experimentation, the patterning of the electrodes and the interconnects was achieved in a one-step process using the fiber laser on a laser engraving system (PLS6MW, ULS, Inc.) with a power of 18 W and a scanning speed of 4 m/s.

Next, a electrode portion 212 of the face layer formed as a result of patterning is removed (iii). What remains is the face layer patterned as electrodes 214,216 positioned on the release liner (iv). Subsequently, a single-sided adhesive sheet 218 may be attached to the electrodes to finish the fabrication process (v). During use, the antenna sticker can be peeled off with the aid of the adhesive sheet (vi).

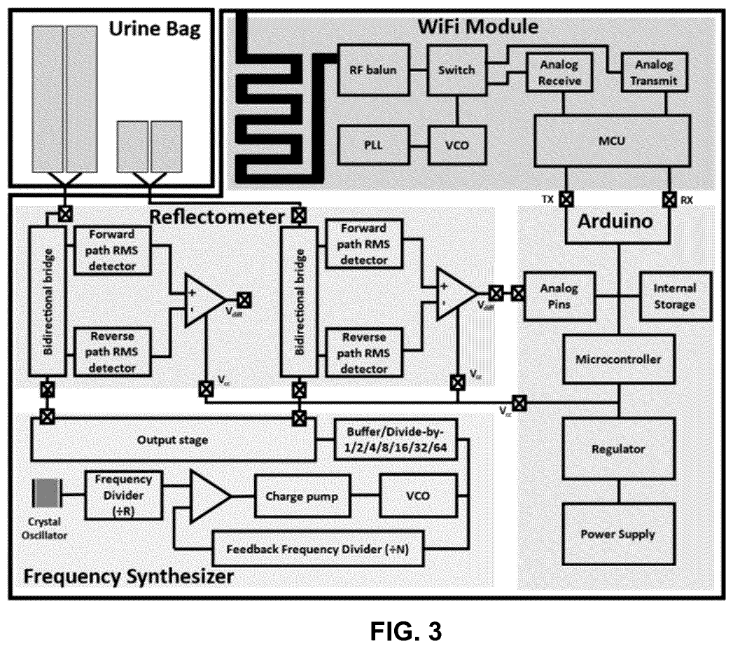

FIG. 3 illustrates a block diagram of electronic components for a reusable portable remote monitoring device. The system is designed as an alternative to the existing portable network analyzers that are heavy, unwieldy, and lack a wireless communication platform. The remote monitoring device may include a network analyzer designed in a modular fashion. The network analyzer may include a frequency synthesizer, two scalar reflectometers, and, in some examples, a microcontroller, and a WiFi module. The frequency synthesizer was realized with ADF4351 which includes a phase locked loop (PLL) operating in tandem with an integrated Voltage Controlled Oscillator (VCO) to provide fundamental output frequencies ranging from 2200 MHz to 4400 MHZ. PLL is a feedback-enabled circuit that detects the phase difference between a reference frequency (f_{ref}) and the required output frequency (f_{vco}) and modifies the output voltage proportionate to the phase difference. The VCO detects this voltage level and modifies the required output frequency in proportion to the voltage. The output of the VCO is fed back to the PLL through a series of frequency counters to establish a feedback mechanism for phase detection and frequency correction through an iterative process. In ADF4351, the PLL is driven by a crystal oscillator which provides an input frequency of 25 MHz. The output of the crystal oscillator is connected to a 10-bit reference counter which can provide relevant frequency division to produce the reference clock to the Phase Frequency Detector (PFD). The phase frequency detector (PFD) takes inputs from the reference counter and the feedback counter and modulates the output voltage of a charge pump proportional to the phase and frequency difference between them. This output voltage modifies the voltage-controlled oscillator frequency of the VCO. To obtain a wide band of frequencies from the fundamental output frequencies, the VCO of ADF4351 is connected to a divide-by-1/-2/-4/-8/-16/-32/-64 circuit which provides frequency sweeping capabilities from 35 MHz to 4400 MHZ.

The signal generated by ADF4351 is fed to two scalar reflectometers (ADL5920 from Analog Devices) to measure the reflection coefficients (S₁₁) from the level detector and the conductivity detector. The scalar reflectometer, ADL5920, includes a bidirectional bridge, a bidirectional detector, and a differential output stage. The bidirectional bridge channels the signal generated by ADF4351 to the electrodes on the urine bag. The bidirectional detector on ADL5920 is comprised of a forward path RMS detector and a reverse path RMS detector that simultaneously measures forward and reverse RMS power levels in the signal path, along with the return loss. Since microcontrollers receive an analog voltage signal input, the detector produces voltages, V_{RMS(F)} and V_{RMS(R)}, proportional to the forward and reflected power in dBm. The differential output stage measures the difference between and produces a voltage proportional to the reflection coefficient (S₁₁) in dB. Since ADL5920 is a wideband scalar reflectometer that operates from 9 kHz to 7 GHz operation with a low insertion loss of <2 dB, combining it with ADF4351 can yield a network analysis platform for accurate S₁₁ measurements in the overlapping frequency range of 35 MHz to 4400 MHz.

To define the required frequency sweep operation for S₁₁ measurements, the frequency synthesizer, and the scalar reflectometer were interfaced with an Arduino Uno microcontroller through I²C communication protocol (See SI document for code). Furthermore, the differential voltage measured by the analyzer module in proportion to the S₁₁ readings are read by the analog pins of the Arduino module. The Arduino performs post-processing of the data to convert the voltage readings to corresponding S₁₁ readings and extracts resonant frequency from the urine level detector and amplitude of the resonant peak from the conductivity detector. Subsequently, the Arduino module converts the values of resonant frequency and amplitude of the resonant peak to urine level and urine conductivity, respectively, using the programmed calibration curve. The collected values are transferred to an ESP8266 WiFi module through the TX and RX pins. The ESP8266 module is a WiFi System on Chip (SOC) with an integrated TCP/IP protocol stack that can provide Arduino access to an 802.11 b/g/n supported WiFi network. With the help of the ESP8266 module, Arduino transmits the urine level and conductivity data over WiFi to the receiving station. The components may be assembled and packaged using a dustproof waterproof IP65 enclosure to form a lightweight portable network analyzer of size 20 cm×10 cm×7 cm. The fully integrated system can be easily transported and attached to urine bags thereby offering efficient cable management and advanced interoperability to manage and coordinate healthcare in a hospital environment.

The components described in FIG. 3 provide non-limiting examples of a possible embodiment. In practice the system may include additional, fewer, or different components than described.

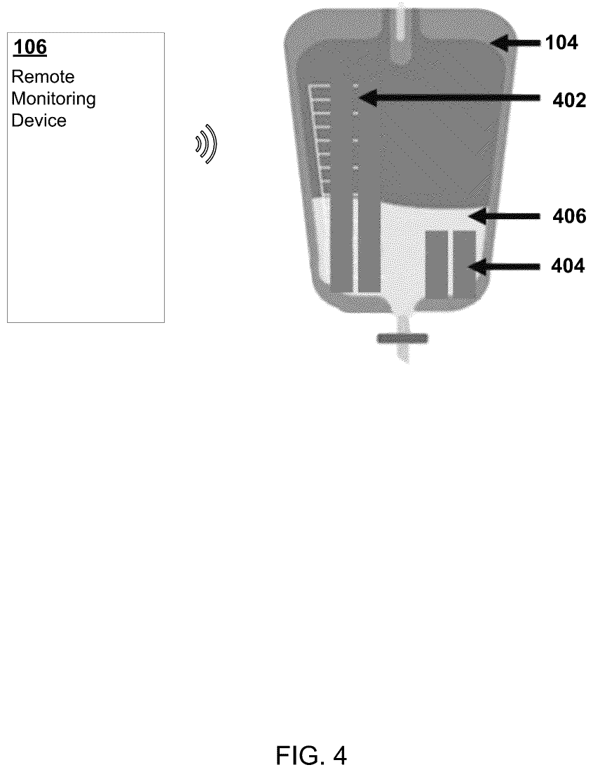

FIG. 4 illustrates an example of the system with a urine container 104 having a pair of 20 cm×2 cm electrodes and a pair of 4 cm×2 cm electrodes forming the level detector antenna 402 and conductivity detector antenna 404, respectively. The remote monitoring device 106 may interrogate the level detector 402 and the conductivity detector 404, respectively.

The remote monitoring device 106 may send a signal in a direction of the fluid container. The remote monitoring device 106 may receive a signal reflected back from the level detector antenna 402 and the conductivity detector antenna 404.

The remote monitoring device 106 may detect a shift in a resonant frequency of the antenna. The level detecting antenna is a resonating structure that includes a pair of 20 cm×2 cm electrodes that forms a parallel plate capacitor. The resonant frequency of the antenna is a function of the fixed inductance (L) of the electrodes and the variable capacitance (C) of the parallel plate capacitor formed by the electrodes as shown in the equation below.

$${f}_{r} = \frac{1}{2 π \sqrt{LC}} \qquad ( Eq 1 )$$

The capacitance of the parallel plate capacitor is dependent on the dielectric constant of the medium surrounding the electrodes as shown in the equation below.

$$C = \frac{l}{π} ln ( 1 + \frac{2 w}{g} ) f \{ {ε}_{a i r} , {ε}_{m} \} \qquad ( Eq 2 )$$

Where (C) is the capacitance and (L) is the inductance of the parallel plate electrodes and (l) is the length of the electrodes, (εₐᵢᵣ) is the relative permittivity of free space, (εₘ) is the dielectric constant of the medium, (w) is the width of the electrodes, and (g) is the gap between the electrodes.

When the bag is empty, the effective dielectric constant of the medium is εₐᵢᵣ (=1). However, when the fluid level in the bag rises, the effective dielectric constant becomes an increasing function of εₘ(˜80), the dielectric constant of the fluid. Consequently, the capacitance between the electrodes increases leading to a shift in the resonant frequency.

The remote monitoring device 106 measure, based on the change in resonant frequency, an amount of fluid in the container. Since the shift in the resonant frequency is proportional to the rise in the fluid level, the amount of fluid in the container can be estimated based on Eq 1 and Eq 2. In our study, 700 MHz corresponded to 0 ml of fluid whereas 200 MHz corresponded to 2000 ml of fluid in a standard sized urine bag.

Furthermore, the remote monitoring device 106 detect a change in an amplitude of a resonant peak of the second antenna. The conductivity detecting antenna utilizes electrodes measuring 4 cm×2 cm, with the smaller dimensions deliberately selected to minimize sensitivity to fluctuations in fluid levels. When the conductivity of the medium increases, the attenuation experienced by the electric field lines between the electrodes increases based on the following equation.

$$α = 2 π f \sqrt{\frac{με}{2} [ \sqrt{1 + {( \frac{σ}{2 π f ε} )}^{2}} - 1 ]} \qquad ( Eq . 3 )$$

Where (α) is the attenuation coefficient, (μ) is the permeability of free space and (f) is the frequency, (σ) is the conductivity, and (ε) is the dielectric constant of the medium.

This leads the dampening of signal propagation and a reduction in the received power through the medium. As a result, the amplitude of the resonant peak decreases with increasing attenuation caused by conductivity rise as shown by the following equation.

$${S}_{11} ∝ {e}^{- 2 α l} \qquad ( Eq . 4 )$$

Where S₁₁ refers to the ratio of the reflected power from the antenna to the transmitted power to the antenna.

The remote monitoring device 106 measure, based on the change in the amplitude of the resonant peak of the second antenna, a conductivity of the fluid. Since the amplitude of the resonant peak is proportional to the rise in conductivity levels in the fluid, the conductivity of urine can be estimated from the amplitude of the resonant peak at any given time. The conductivity detecting antenna provides a linear sensitivity of 1 dB/S/m within the physiologically relevant urine conductivity range of 10-40 mS/cm.

To optimize the operating frequency range and to study the effect of the volume and conductivity of urine 406 on the metalized stickers, systematic simulations were performed in CST Microwave Studio.

The length of the level detector was chosen as 20 cm based on the typical dimensions of commercially available urine bags to obtain maximum sensitivity to urine levels. On the other hand, the length of the conductivity detector was set to 4 cm to ensure insensitivity to the variations in urine levels. Both the level detector and the conductivity detector were separated from a conductive solution with a polymer layer of thickness 0.1 mm which was obtained from commercially available bags.

The simulations demonstrate the electric field distribution and the current flow associated with the level and conductivity detector's antennas. To identify the operating frequency range of the level detector, the level of the conductive solution was varied from 0 ml to 2000 ml and the S11 characteristics were plotted for the maximum and minimum values of urine conductivity.

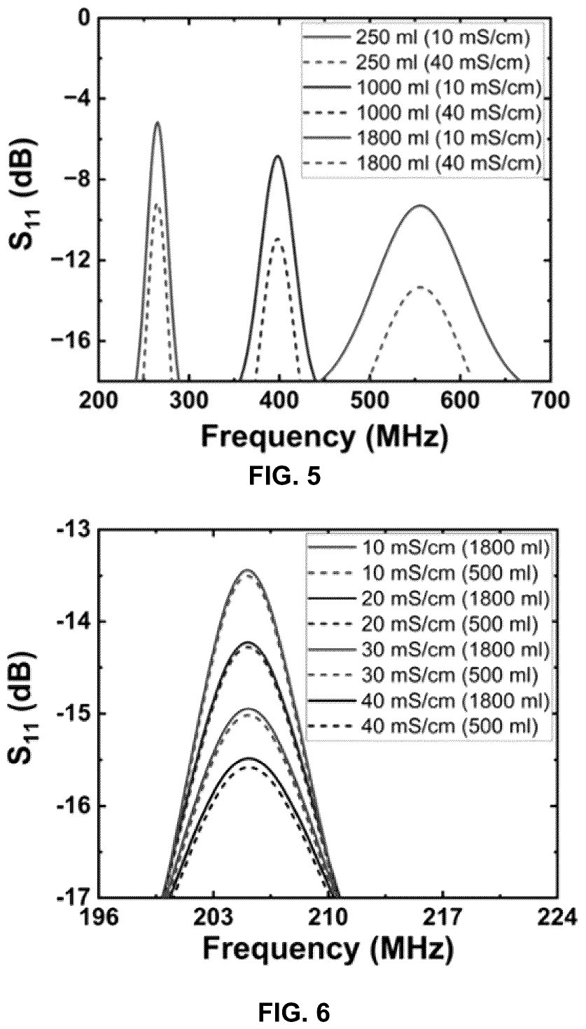

FIG. 5 illustrates results demonstrating a shift in the resonant frequency characteristics for different values of fluid volume. As illustrated in FIG. 5, the resonant frequency of the level detector decreased from 650 MHz to 250 MHz as the volume of the conductive solution was increased from 0 ml to 2000 ml, providing an excellent sensitivity of 200 kHz/ml for level detection. Furthermore, the resonant frequency showed negligible deviation with respect to the conductivity of the solution indicating the stability of the measurements to varying electrical properties of the solution.

After identifying the operating frequency range of the level detector, the matching network of the conductivity detector was capacitively tuned to ensure that the resonant frequency of the conductivity detector does not overlap with the frequency band of the level detector.

FIG. 6 illustrates results demonstrating the shift in the amplitude of the resonant peak for different values of urine conductivity. The simulation results shown in FIG. 6 indicate that the resonant frequency can be tuned to <250 MHz to avoid spectral overlap. Moreover, the simulations demonstrate that the resonant peak of the S11 characteristics shows excellent sensitivity of 1 dB/S/m in the conductivity range of 10-40 mS/cm. In addition, when the volume was changed from 500 ml to 1800 ml, S11 characteristics showed negligible change indicating the ability of the conductivity detector to provide stable readings regardless of the level of urine in the bag.

To identify the optimum dimensions for the electrodes that provide maximum readability to the urine level detector and the highest sensitivity to the urine conductivity detector, a series of simulations were performed in CST Microwave Studio. Q-factor is an excellent indicator of the readability of the electrodes as it represents the sharpness of resonance expressed as a function of the resonant frequency (f₀) and bandwidth (Δf) using the following equation:

$$Q = \frac{{f}_{0}}{Δ f} \qquad ( Eq . 5 )$$

As Q-factor is a function of the gap between the electrodes (g) and the width of the electrodes (w), the simulations were performed by varying g from 0 mm to 5 mm and w from 0 mm to 50 mm in order to identify the optimum values of g and w that provide maximum Q-factor. It was found that when the width of the electrodes was lower than 10 mm and greater than 30 mm, the Q-factor was <100 for all values of g. At very low values of w, the density of the electric field is too low whereas, at very high values of w, the attenuation between the electrodes increases drastically leading to a decrease in the Q factor. However, when w was in the range of 10 mm and 30 mm Q-factor increased significantly to values above 100 peaking at 400 for w=20 mm. At w=20 mm, the Q factor was <200 for values of g above 3 mm. This can be attributed to the increase in attenuation experienced by the electric field lines caused by the increased distance between the electrodes. In contrast, lower values of g provided higher Q-factor values due to diminished attenuation. For w=20 mm, the maximum Q-factor was attained at g=2 mm. Thus, the optimum dimensions of width and gap for the level detector were chosen to be 20 mm and 2 mm, respectively.

Similarly, to obtain the optimum width and gap that provides maximum sensitivity to the conductivity detector, the values of g and w were varied from 0 mm to 5 mm and 0 mm to 50 mm, respectively, and the overall change in the resonant peak (ΔS₁₁) was plotted. It was found that ΔS₁₁ lower than 10 dB when w is lower than 10 mm. However, when w was increased from 10 mm, ΔS₁₁ increased steadily and attained a peak value at w=20 mm followed by a gradual decline. Furthermore, within various values of g at w=20 mm, g=2 mm provided the maximum ΔS₁₁. From this set of simulations, we identified w=20 mm and g=2 mm as the optimum dimensions for the design of the conductivity detector.

While the experimental and simulation results were based on urine activity monitoring, the system, sticker, and methods described herein may be applied in other settings where fluid levels inside flexible containers is desired.

A second action may be said to be “in response to” a first action independent of whether the second action results directly or indirectly from the first action. The second action may occur at a substantially later time than the first action and still be in response to the first action. Similarly, the second action may be said to be in response to the first action even if intervening actions take place between the first action and the second action, and even if one or more of the intervening actions directly cause the second action to be performed. For example, a second action may be in response to a first action if the first action sets a flag and a third action later initiates the second action whenever the flag is set.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While various embodiments have been described, it will be apparent to those of ordinary skill in the art that many more embodiments and implementations are possible. Accordingly, the embodiments described herein are examples, not the only possible embodiments and implementations.

What is claimed is:

1. A system comprising:
a sticker configured to attach to a fluid container, the sticker comprising a metal layer shaped as an antenna;
a processor, the processor configured to:
cause a network analyzer to send a signal in a direction of the fluid container;
detect a shift in a resonant frequency of the antenna; and
measure, based on the change in resonant frequency, an amount of fluid in the container.

2. The system of claim 1, wherein the antenna comprises parallel electrodes placed adjacent to each other.

3. The system of claim 1, wherein the sticker is attached to an outside of the container.

4. The system of claim 1, wherein the fluid container is a flexible fluid bag.

5. The system of claim 1, wherein the sticker comprises an adhesive layer which covers the metal layer and attaches the metal layer to the fluid container.

6. The system of claim 1, wherein the antenna is a first antenna, where the system further comprises:
a second antenna,
wherein the processor is further configured to:
detect a change in an amplitude of a resonant peak of the second antenna; and
measure, based on the change in the amplitude of the resonant peak of the second antenna, a conductivity of the fluid.

7. The system of claim 6, wherein the second antenna comprises parallel plate resonators.

8. The system of claim 6, wherein the first antenna and the second antenna share an adhesive layer, which attaches the first antenna and the second antenna to the fluid container.

9. The system of claim 6, wherein the first antenna comprises parallel plate resonators and the second antenna comprises parallel plate resonators, wherein the parallel plate resonators of the first antenna are longer than the parallel plate resonators of the second antenna.

10. An antenna sticker, comprising:
a release layer;
an antenna layer comprising, a first adhesive layer, a metal layer, and a substrate layer between the first adhesive layer and metal layer, where the first adhesive layer, the metal layer, and the substrate layer are in the shape of an antenna; and
a second adhesive layer covering at least a portion of the antenna layer and at least a portion of the release layer,
wherein the antenna layer and second adhesive layer separate from the release layer in response to removing the second adhesive layer from the release layer.

11. The antenna sticker of claim 10, wherein the antenna comprises parallel plate resonators.

12. The antenna sticker of claim 10, wherein the antenna comprises a first antenna and a second antenna.

13. The antenna sticker of claim 12, wherein the first antenna comprises parallel plate resonators and the second antenna comprise parallel plate resonators.

14. The antenna sticker of claim 13, wherein the parallel plate resonators of the second antenna are shorter than the parallel plate resonators of the first antenna.

15. A method, comprising:
providing a metalized paper comprising a release layer, a first adhesive layer, a substrate layer, and a metal face layer;
laser cutting an outline of an antenna into the metal face layer, substrate layer, and first adhesive layer;
removing, from the release layer, a portion of the metal face layer, a portion of the substrate layer, and a portion of the first adhesive layer leaving the antenna attached to the release layer; and
applying an adhesive layer over the antenna so that the adhesive layer adheres to the antenna and the release layer.

16. The method of claim 15, wherein the antenna comprises parallel electrodes.

17. The method of claim 15, wherein the antenna comprises a first antenna and a second antenna.

18. The method of claim 17, wherein the first antenna comprises parallel electrodes and the second antenna comprises parallel electrodes.

19. The method of claim 18, wherein the parallel electrodes of the first antenna are shorter than the parallel electrodes of the second antenna.
